(19) United States
(12) Patent Application Publication
(10) Pub. No.: US 2003/0153095 A1
(43) Pub. Date: Aug. 14, 2003

(54) Mixture experiment design method and system

(75) Inventor: James Norman Cawse, Pittsfield, MA (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(73) Assignee: General Electric Company

(21) Appl. No.: 10/036,227

(22) Filed: Jan. 4, 2002

(51) Int. Cl. G06F 19/00; G01N 33/48; G01N 33/50; G01N 33/543

(52) U.S. Cl. 436/518; 702/19; 702/22

(57) ABSTRACT

An experimental space is determined comprising n factors and a first factor in M number of factor level intervals and in a range of Aₘᵢₙ to Aₘₐₓ where A is a proportion of the factor level to total factor levels. An experiment is conducted on the first factor sampled in a range of levels determined according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1))). A system comprises a reactor for effecting a CHTS method on an experimental space to produce results and a programmed controller for the reactor that defines an experimental space comprising a lattice of points representing increments of reaction factor levels from a minimum level value to a maximum level value according to the relationship (Aₘᵢₙ+(Aₘₐₓ−Aₘᵢₙ)/(n(M−1))) to (Aₘₐₓ−Aₘᵢₙ)/(n(M−1))) where M is a number of intervals for the factor levels of the range, n is a number of mixture components and A is a proportion of the factor level to total factor levels.

### BACKGROUND OF THE INVENTION

The invention relates to a mixture experiment design method and system.

Mixture experiments relate to the testing of multifactor gradient combinations. In a mixture experiment, response is assumed to depend only on relative proportions of the ingredients or factors present in the mixture and not on an amount of the mixture. In a mixture experiment, if the total amount is held constant and the value of the response changes when changes are made in the relative proportions of the ingredients or factor levels making up the mixture, then the behavior of the response can be said to be a measure of the joint blending property of the ingredients or factors in the mixture. John A. Cornell, Experiments with Mixtures, 2^{nd} Ed., p. 13, 1990.

One type of mixture experiment involves preparation of a gradient array. For example, the development of materials such as phosphors for lighting applications can involve the testing of gradient arrays of materials by a methodology called combinatorial high throughput screening (CHTS). Sun, Combinatorial Search for Advanced Luminescence Materials, Biotechnology and Bioengineering (Combinatorial Chemistry), vol. 61, 4, pp. 193, 201 (1999). The methodology of CHTS as applied to materials has evolved from combinatorial organic synthesis (COS). COS is a high throughput screening (HTS) method that uses systematic and repetitive synthesis to produce libraries of diverse pharmaceutical molecular entities formed from sets of chemical “building blocks.” COS applies automation and miniaturization to produce the libraries through successive stages, each of which produces a chemical modification of an existing molecule of a preceding stage. For example, Pirrung et al., U.S. Pat. No. 5,143,854 discloses a technique for generating arrays of peptides and other molecules using light-directed, spatially-addressable synthesis techniques.

For a number of reasons, it is difficult to apply the methodology of CHTS to catalyzed chemical reactions. First chemical reactions, particularly industrial catalyzed chemical reactions, can involve large numbers of factors and require investigation of enormous numbers of factor levels (settings). For example, even a simple commercial process can involve five or six critical factors, each of which can be set at 2 to 20 levels. A complex homogeneous catalyst system may involve two, three, or even more metal cocatalysts that can synergistically combine to improve the overall rate of the process. These cocatalysts can be chosen from a large list of candidates. Additional factors can include reactants and processing conditions. The number of tertiary, 4-way, 5-way or n-way factor combinations can rapidly become extremely large, depending on the number of levels for each factor.

Another problem is that catalyzed chemical reactions are unpredictable. T. E. Mallouk et al. in Science, 1735 (1998) shows that effective ternary combinations can exist in systems in which no binary combinations are effective. Additionally, a reaction may involve a highly dimensional space. As illustrated in FIG. 1, a high dimensional material system can be characterized by multiple phases with different properties. Desired properties may appear in only a small fraction of the phases. The property of interest is not part of a simple continuous function of factors. This contrasts with assumptions of conventional design of experiments (DOE). In conventional DOE, response is a linear function of the factors, their interactions, and perhaps quadratic terms. In combinatorial search, a response may be a phase space with irregular boundaries or a highly jagged landscape. Accordingly, a simple CHTS experiment on a catalyzed chemical space may require search of enormous numbers of factor level combinations to find a handful of “leads,” i.e., combinations that may lead to commercially valuable applications. However, an exhaustive search of the space can be prohibitively time-consuming and expensive.

One answer to the problem of extensive exhaustive test searching is to carefully select and organize the experimental space of the system. Since testing is expensive, any reduction in the number of samples necessary to effectively sample an experimental space is important. However in this respect, the challenge is to define a reasonable number of samples to effectively investigate a particular experimental space to provide meaningful results. There is a need for an experimental protocol to specify arrangements of formulations and processing conditions to reduce the number of samples necessary to reliably and effectively detect positive interactions of formulation and processing variables of a particular experimental space.

### BRIEF SUMMARY OF THE INVENTION

The present invention relates to an experimental design strategy for evaluating mixture systems with complex physical, chemical and structural requirements. According to the strategy, an experimental space is defined and factors of the space are sampled in a manner that permits a reliable and efficient investigation of highly complex mixture systems.

In the method, an experimental space is determined comprising n factors and a first factor in M number of factor level intervals and in a range of Aₘᵢₙ to Aₘₐₓ where A is a proportion of the factor level to total factor levels. An experiment is conducted on the first factor sampled in a range of levels determined according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1))).

In another embodiment, the invention is a method for defining a reduced set of samples for an experimental space and conducting an experiment on the samples, comprising determining an experimental space comprising n factors in M number of factor level intervals and in a range of Aₘᵢₙ to Aₘₐₓ where A is a proportion of a factor level to total factor levels, specifying new factor level ranges for each factor according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1))), selecting samples of combinations of factors in a set of M−1 evenly spaced levels within the specified levels and conducting an experiment on the samples.

Further, the invention relates to a system for conducting an experiment. The system comprises a reactor for effecting a CHTS method on an experimental space to produce results and a programmed controller for the reactor that defines an experimental space comprising a lattice of points representing increments of reaction factor levels from a minimum level value to a maximum level value according to the relationship (Aₘᵢₙ+(Aₘₐₓ−Aₘᵢₙ)/(n(M−1))) to (Aₘₐₓ−(Aₘₐₓ−Aₘᵢₙ)/(n(M−1))) where M is a number of intervals for the factor levels of the range, n is a number of mixture components and A is a proportion of the factor level to total factor levels.

In another embodiment, the invention is a system for conducting an experiment, comprising a reactor for effecting a CHTS method on an experimental space to produce results and a programmed controller for the reactor for inputing experimental space information comprising n factors in M number of factor level intervals and in a range of Aₘᵢₙ to Aₘₐₓ where A is a proportion of a factor level to total factor levels, specifying new factor level ranges for each factor according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1))) and selecting samples of combinations of factors in a set of M−1 evenly spaced levels within the specified levels for charge to the reactor.

### BRIEF DESCRIPTION OF THE DRAWING

FIG. 1 is a histograph showing a two-dimensional response surface.

FIG. 2 is a schematic representation of a system and method for conducting a CHTS experiment.

FIG. 3 is a schematic representation of a CHTS method.

FIGS. 4 to 9, 11 and 14 are schematic representations of experimental spaces.

FIGS. 10, 12, and 13 are contour representations of experimental results.

### DETAILED DESCRIPTION OF THE INVENTION

The object of a CHTS experiment is to determine purposeful input variables (experimental space) for a process or system so that reasons for changes in an output response can be observed. The steps of a CHTS methodology can be broken down into generic operations including selecting chemicals to be used in an experiment; introducing the chemicals into a formulation system (typically by weighing and dissolving to form stock solutions); combining aliquots of the solutions into formulations or mixtures in a geometrical array (typically by the use of a pipetting robot); processing the array of chemical combinations into products; and analyzing properties of the products. Results from the analyzing step can be used to compare properties of the products in order to discover “leads”—materials whose properties indicate commercial potential.

Typically, a CHTS experiment is characterized by parallel reactions at a micro scale. In one aspect, CHTS can be described as a method comprising (A) an iteration of steps of (i) selecting a reactant, catalyst or condition set; (ii) reacting the set; and (iii) evaluating products of the reacting step; and (B) reiterating step (A) wherein a successive reactant, catalyst or condition set selected for a step (i) is chosen as a result of an evaluating step (iii) of a preceding iteration.

In another CHTS method, a multiplicity of tagged reactants is subjected to an iteration of steps of (A) (i) simultaneously reacting the reactants, (ii) identifying a multiplicity of tagged products of the reaction and (B) evaluating the identified products after completion of a single or repeated iteration (A). A CHTS can utilize advanced automated, robotic, computerized and controlled loading, reacting and evaluating procedures.

In one CHTS method, the reactant or catalyst is at least partially embodied in a liquid and the steps of effecting the CHTS method comprise contacting the reactant or catalyst with an additional reactant at least partially embodied in a gas, wherein the liquid forms a film having a thickness sufficient to allow a reaction rate that is essentially independent of a mass transfer rate of additional reactant into the liquid to synthesize products that comprise the results.

According to the present invention, an experimental design strategy is provided for CHTS evaluation of a simplex with complex physical, chemical and structural requirements. According to the invention, spherical sampling concepts are applied to define experimental space for a reiterated CHTS simplex method. A simplex method is an experiment on a continuous and regular experimental space such as a mixture. In a simplex system, factors are constrained to sum to a constant, usually 1.00 or 100%. The simplex can be represented as a regular sided figure with k +1 vertices in k dimensions. Thus for k=2, the simplex design is an equilateral triangle and for k=3, it is a regular tetrahedron.

According to the invention, a successive experimental space for a reiterated CHTS experiment is defined by a spherical sampling concept. In the concept, covering spheres are defined to extend only to edges of the space and not beyond. Features of the invention will become apparent from the drawings and following detailed discussion, which by way of example without limitation describe preferred embodiments of the present invention.

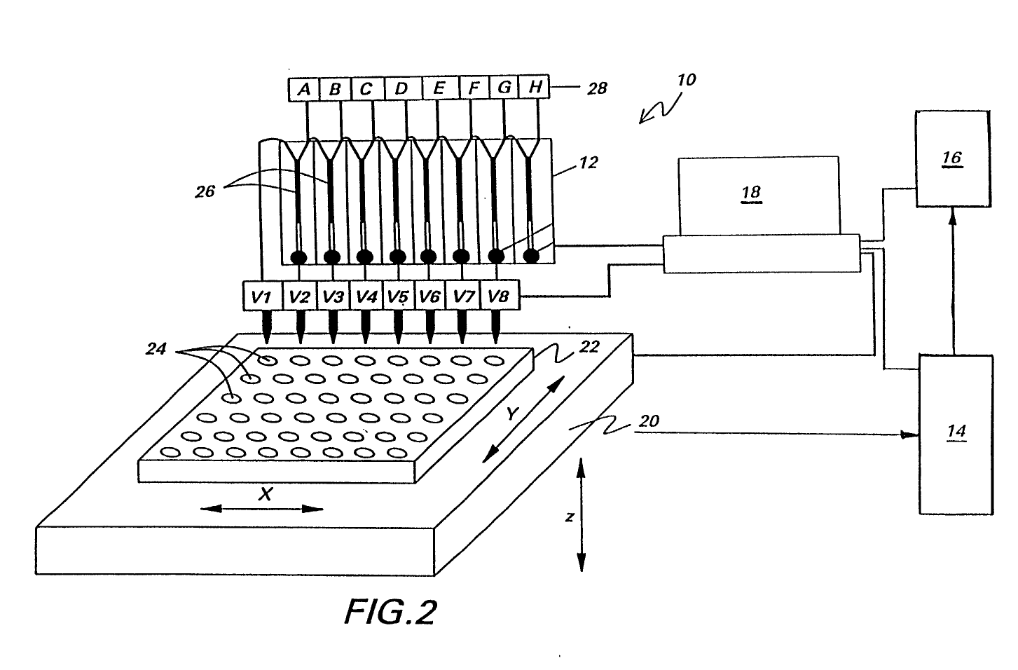

FIG. 2 is a schematic representation of a system 10 and method for conducting a CHTS experiment. FIG. 2 shows system 10 including dispensing assembly 12, reactor 14, detector 16 and controller 18. Further shown is X-Y-Z robotic positioning stage 20, which supports array plate 22 with wells 24. The dispensing assembly 12 includes a battery of pipettes 26 that are controlled by controller 18. X-Y-Z robotic positioning stage 20 is controlled by controller 18 to position wells 24 of the array plate 22 beneath displacement pipettes 26 for delivery of test solutions from reservoirs 28.

Controller 18 can include a data base repository for storing specified design inputs from an experimenter. The controller can include algorithms or programs for specifying the lattice-defined design as well. The controller 18 also controls aspiration of precursor solution into the battery of pipettes 26 and sequential positioning of the wells 24 of array plate 22 so that a prescribed stoichiometry and/or composition of reactant and/or catalyst can be delivered to the wells 24. By coordinating activation of the pipettes 26 and movement of plate 22 on the robotic X-Y-Z stage 20, a library of materials can be generated in a two-dimensional array for use in the CHTS method. Also, the controller 18 can be used to control sequence of charging of sample to reactor 14 and to control operation of the reactor 14 and the detector 16. Controller 18 can be a computer, processor, microprocessor or the like.

Controller 18 specifies the spaces according to the requirements of the experiment as may be input by an experimenter and the spaces are then translated into loading specifications for array plates 33. Then controller 18 controls the operation of pipettes 26 and stage 20 according to the specifications to deliver reactant and/or catalyst to the wells 34 of plates 22. Additionally, the controller 18 controls the sequence of charging array plate 22 into the reactor 14, which is synchronized with operation of detector 16. Detector 16 detects products of reaction in the wells 24 of array plate 22 after reaction in reactor 14. Detector 16 can utilize chromatography, infra red spectroscopy, mass spectroscopy, laser mass spectroscopy, microspectroscopy, NMR or the like to determine the constituency of each reaction product. The controller 18 uses data on the sample charged by the pipettes 26 and on the constituency of reaction product for each sample from detector 16 to correlate a detected product with at least one varying parameter of reaction.

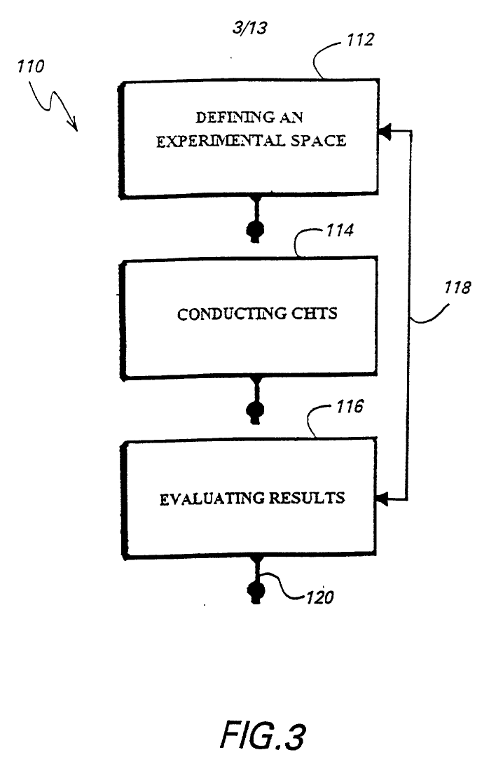

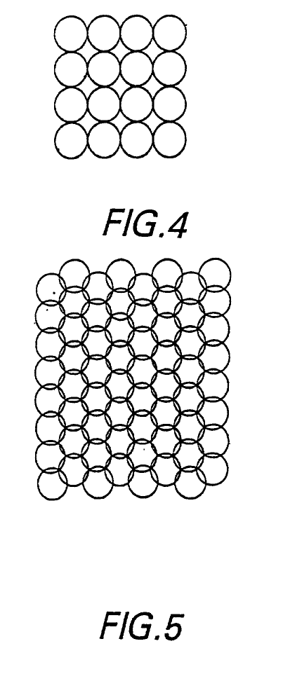

FIG. 3 is a schematic representation of a method for operating the system shown in FIG. 2. The method 110 is an improved protocol for searching a high dimensional experimental space to select a best case set of levels of factors of a reaction. The method 110 comprises first defining 112 an experimental space comprising a lattice of points representing increments of reaction factor levels. A lattice is a regular array of n-dimensional spheres in n-space. The lattices can be either a packing lattice, in which spheres representing effectively sampled space just touch (FIG. 4) or a covering lattice, in which single space spheres overlap to cover an entire space (FIG. 5). A covering lattice represents a minimum distance possible from the centers of the spheres (sampling points) to any other point in the experimental space. A covering lattice provides a high covering efficiency where covering efficiency is the number of spheres of a given radius required to completely cover an experimental space. Inefficient lattices are characterized by excessive sphere overlap.

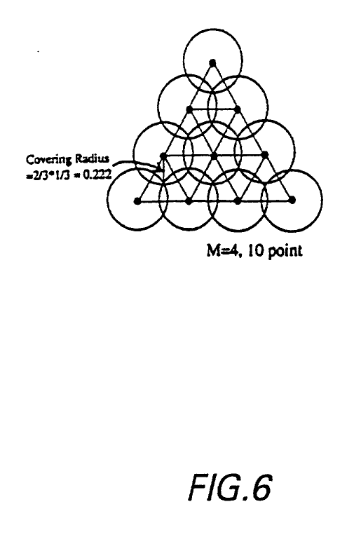

A typical simplex gradient design can be specified by points that lie on regular intervals on A, B, and C (D, E . . . ) axes as shown in FIG. 6. The number of intervals in the gradient on each axis and the increment size is a (1/(M−1)) fraction of the gradient range. If each point represents a sample of the space closest to it, covering of space by each point can be represented by circumscribed spheres of radius such that the entire simplex (triangle, tetrahedron, . . . ) is covered. The covering radius of the spheres is equal to ⅔ of the gradient interval for the FIG. 6 three component case. Accordingly, a first experimental space can be defined 112 and a first CHTS experiment conducted 114 on the first space. Results from the conducting 114 step are evaluated 116 to identify a first best result. A second space can then be defined 112 according to a simplex gradient design around the first best result but in narrower increments than the first design. A second CHTS experiment is then conducted 114 according to the space defined around the first best result. The results from the second experiment are then evaluated 116 to identify a second best result. The iterations of the method are repeated 118 until a lead is identified 120.

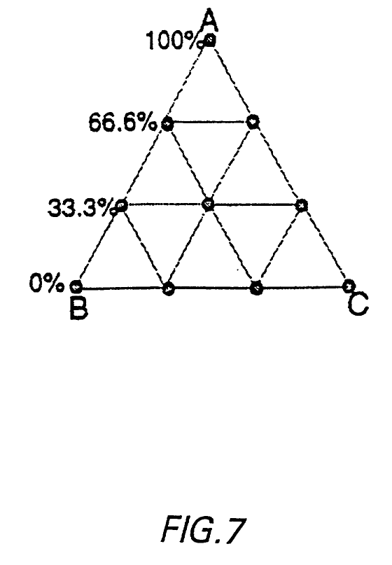

According to the invention, a ternary system experimental space can be defined by first identifying the system in terms of A, B, C factors and selecting a number of intervals M for the factors that can be used in a gradient study as illustrated by FIG. 7. The simplex gradient design can be specified by points that lie on regular intervals on A, B, and C axes shown in FIG.7. The number of intervals in the gradient on each axis is M and the interval step size is (1/(M−1)). Because of the simplex constraint, spaces for n=3, 4, . . . are represented by triangles, tetrahedra or by a space that would correspond to a hyperpolyhedra. If each point is considered to be a sample of a closed space, each sampling can be represented by a circumscribed sphere of radius such that the entire simplex (triangle, tetrahedron, . . . ) is covered.

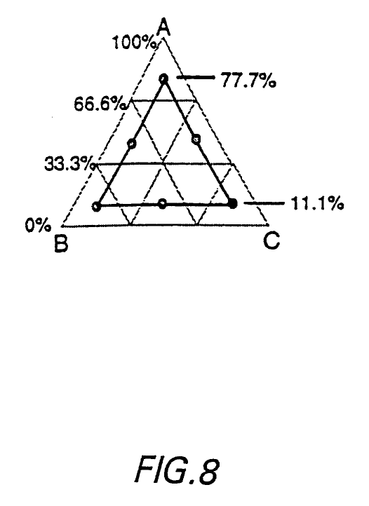

In FIG. 6, sampling spheres are represented as having centers that extend to the edge of the space. In FIG. 6, a significant fraction of spherical volume is outside the triangle. (In a two-dimensional representation, a “sphere” is a circle and its “volume” is its area.) The fraction of spherical volume outside the triangle has no physical meaning. That is, there is no physically possible combination of A, B, and C that can be represented by a point outside the triangle. A more efficient sampling scheme is shown in FIG. 8. FIG. 8 represents an inventive protocol wherein the sampling spheres are represented as having centers in a contracted area that spheres only extend minimally outside the edge of the space. In the embodiment illustrated in FIG. 8, the contracted sampling area for each of A, B, and C is defined by the relationship (100/(3(M−1))) to (100-200/(3(M−1))). In a quaternary case, new ranges for factor levels can be calculated by the relationship (100/(4(M-I))) to (100-200/(4(M-I))).

The FIG. 8 type of contracted covering can be applied to systems of higher dimension. The number of points required for ternary, quaternary, and centenary systems is summarized by TABLE 1.

TABLE 1

| | | Ternary | | Quaternary | | Pentanary | |
|---|---|---|---|---|---|---|---|
| | | | Sphere | | Sphere | | Sphere |
| | | Simplex | Packing | Simplex | Packing | Simplex | Packing |
| Gradient | M | Gradient | Covering | Gradient | Covering | Gradient | Covering |
| 50.00% | 3 | 6 | 3 | 10 | 4 | 30 | 10 |
| 33.33% | 4 | 10 | 6 | 20 | 10 | 70 | 30 |
| 25.00% | 5 | 15 | 10 | 35 | 20 | 140 | 70 |
| 20.00% | 6 | 21 | 15 | 56 | 35 | 252 | 140 |
| 12.50% | 9 | 45 | 36 | 165 | 120 | 990 | 660 |
| 11.11% | 10 | 55 | 45 | 220 | 165 | 1430 | 990 |
| 10.00% | 11 | 66 | 55 | 286 | 220 | 2002 | 1430 |
| 8.33% | 13 | 91 | 78 | 455 | 364 | 3840 | 2730 |
| 6.25% | 17 | 153 | 136 | 969 | 816 | 9690 | 7752 |
| 5.00% | 21 | 231 | 210 | 1771 | 1540 | 21252 | 17710 |

| | | | | | |
|---|---|---|---|---|---|
| M * (M + 1)/2 | (M − 1) * M/2 | $\frac{M * ( M + 1 ) * ( M + 2 )}{6}$ | $\frac{( M - 1 ) * M * ( M + 2 )}{6}$ | $\frac{\begin{matrix}M * ( M + 1 ) * \\ ( M + 2 ) * ( M + 3 )\end{matrix}}{24}$ | $\frac{\begin{matrix}( M - 1 ) * M * \\ ( M + 2 ) * ( M + 3 )\end{matrix}}{24}$ |

The contracted sampling area requires a reduced number of sampling points to sample the same experimental space. The savings in points becomes more significant in higher dimensional experimental space. A general case is defined by considering a system with n factors. For any system with n factors, the ranges will be (100/(n(M−1))) to (100-200/(n(M−1))).

In one embodiment, the invention is applied to study a process for preparing diaryl carbonates. Diaryl carbonates such as diphenyl carbonate can be prepared by reaction of hydroxyaromatic compounds such as phenol with oxygen and carbon monoxide in the presence of a catalyst composition comprising a Group VIIIB metal such as palladium or a compound thereof, a bromide source such as a quaternary ammonium or hexaalkylguanidinium bromide and a polyaniline in partially oxidized and partially reduced form. The invention can be applied to screen for a catalyst to prepare a diaryl carbonate by carbonylation.

Various methods for the preparation of diaryl carbonates by a carbonylation reaction of hydroxyaromatic compounds with carbon monoxide and oxygen have been disclosed. The carbonylation reaction requires a rather complex catalyst. Reference is made, for example, to Chaudhari et al., U.S. Pat. No. 5,917,077. The catalyst compositions described therein comprise a Group VIIIB metal (i.e., a metal selected from the group consisting of ruthenium, rhodium, palladium, osmium, iridium and platinum) or a complex thereof.

The catalyst material also includes a bromide source. This may be a quaternary ammonium or quaternary phosphonium bromide or a hexaalkylguanidinium bromide. The guanidinium salts are often preferred; they include the ∀, T-bis(pentaalkylguanidinium)alkane salts. Salts in which the alkyl groups contain 2-6 carbon atoms and especially tetra-n-butylammonium bromide and hexaethylguanidinium bromide are particularly preferred.

Other catalytic constituents are necessary in accordance with Chaudhari et al. The constituents include inorganic cocatalysts, typically complexes of cobalt(II) salts with organic compounds capable of forming complexes, especially pentadentate complexes. Illustrative organic compounds of this type are nitrogen-heterocyclic compounds including pyridines, bipyridines, terpyridines, quinolines, isoquinolines and biquinolines; aliphatic polyamines such as ethylenediamine and tetraalkylethylenediamines; crown ethers; aromatic or aliphatic amine ethers such as cryptanes; and Schiff bases. The especially preferred inorganic cocatalyst in many instances is a cobalt(II) complex with bis-3-(salicylalamino)propylmethylamine.

Organic cocatalysts may be present. These cocatalysts include various terpyridine, phenanthroline, quinoline and isoquinoline compounds including 2,2′:6′,2″-terpyridine, 4-methylthio-2,2′:6′,2″-terpyridine and 2,2′:6′,2″-terpyridine N-oxide,1,10-phenanthroline, 2,4,7,8-tetramethyl-1,10-phenanthroline, 4,7-diphenyl-1,10, phenanthroline and 3,4,7,8-tetramethy- 1,10-phenanthroline. The terpyridines and especially 2,2′:6′,2″-terpyridine are preferred.

Another catalyst constituent is a polyaniline in partially oxidized and partially reduced form.

Any hydroxyaromatic compound may be employed. Monohydroxyaromatic compounds, such as phenol, the cresols, the xylenols and p-cumylphenol are preferred with phenol being most preferred. The method may be employed with dihydroxyaromatic compounds such as resorcinol, hydroquinone and 2,2-bis(4-hydroxyphenyl)propane or “bisphenol A,” whereupon the products are polycarbonates.

Other reagents in the carbonylation process are oxygen and carbon monoxide, which react with the phenol to form the desired diaryl carbonate.

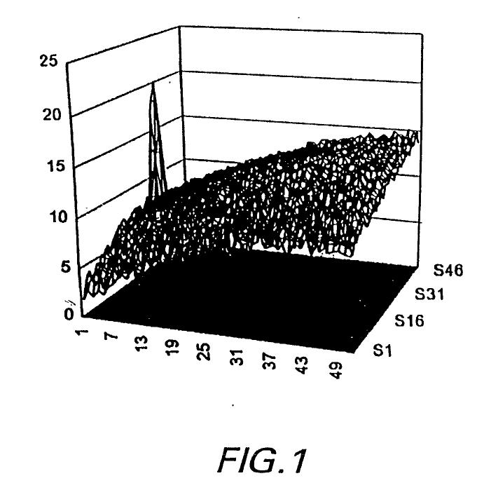

As an example, if the method and system of FIG. 1 is applied to study a carbonylation catalyst and/or to determine optimum carbonylation reaction conditions, the detector 16 analyzes the contents of the well for carbonylated product. In this case, the detector 16 can use Raman spectroscopy. The Raman peak is integrated using the analyzer electronics and the resulting data can be stored in the controller 18. Other analytical methods may be used—for example, Infrared spectrometry, mass spectrometry, headspace gas-liquid chromatography and fluorescence detection.

The following Examples are illustrative and should not be construed as a limitation on the scope of the claims unless a limitation is specifically recited.

### EXAMPLE 1

This example illustrates an identification of an active and selective catalyst for the production of aromatic carbonates. The procedure identifies the best cocatalyst from a complex chemical space, where the chemical space is defined as an assemblage of ratios of combinations of certain Group IVb, Group VIb, and Lanthanide Group metal complexes. In this experiment, the factors are components of the carbonylation catalyst as listed in TABLE 2.

TABLE 2

| Group IVb complex | Group VIb complex | Lanthanide complex |
|---|---|---|
| Ti(acac)2 | Cr(acac)3 | Ce(acac)3 |

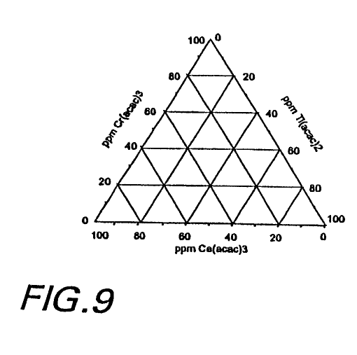

The components are sampled according to a ternary gradient mixture experiment (a simplex) at 20% steps as shown in FIG. 9. In FIG. 9, each line intersection represents one mixture to be tested. There are 21 mixtures to be tested in the system. The mixtures are made up in phenol solvent according to TABLE 3.

TABLE 3

| | | |
|---|---|---|
| Primary Catalyst: | Pd(acac)2 | 25 ppm |
| Anion Cocatalyst | (Et3N)Br | 800 ppm |

The mixture samples with concentrations of each component are given in the rows of TABLE 4.

TABLE 4

| Ti(acac)2 | Cr(acac)3 | Ce(acac)3 | TON |
|---|---|---|---|
| 0 | 0 | 100 | 710 |
| 0 | 20 | 80 | 872 |
| 0 | 40 | 60 | 891 |
| 0 | 60 | 40 | 902 |
| 0 | 80 | 20 | 857 |
| 0 | 100 | 0 | 800 |
| 20 | 0 | 80 | 808 |
| 20 | 20 | 60 | 1131 |
| 20 | 40 | 40 | 1302 |
| 20 | 60 | 20 | 1148 |
| 20 | 80 | 0 | 802 |
| 40 | 0 | 60 | 751 |
| 40 | 20 | 40 | 1270 |
| 40 | 40 | 20 | 1328 |
| 40 | 60 | 0 | 827 |
| 60 | 0 | 40 | 797 |
| 60 | 20 | 20 | 1173 |
| 60 | 40 | 0 | 788 |
| 80 | 0 | 20 | 739 |
| 80 | 20 | 0 | 840 |
| 100 | 0 | 0 | 811 |

Total volume of each catalyst mixture is 1.0 ml. A 25 microliter aliquot of each mixture is dispensed into a 2 ml reaction vial, forming a film on the vial bottom. The vials are subjected to reaction at 1000 psi pressure and 100° C. for 120 min. At the end of the reaction time, the system is cooled and depressurized. The contents of each vial are analyzed for diphenyl carbonate product. A turnover number (TON) for each reaction is calculated as mols of diphenylcarbonate/mols of primary catalyst. Results are given in the column TON of TABLE 4.

Analysis of the data by regression techniques for mixtures, (Cornell, “Experiments with Mixtures,” John Wiley and Sons, 1990, pp. 93-98) yields the following model:

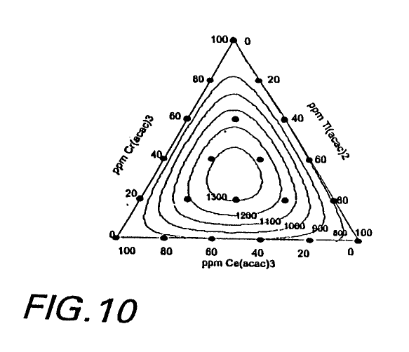

TON=+8.0*A+7.85*B+7.46*C+0.0115*A*B+5.55E-004*A*C +0.0534**B*C+0.0137*A*B*C,

where A=ppm Ti(acac)₂; B=ppm Cr(acac)₃; and C=ppm Ce(acac)₃; and “*” denotes multiplication. This model is a special cubic Scheffe model (Ref: Cornell, Chapter 1), which is a preferred choice for modeling a mixture region containing a maximum. The model standard deviation is 33 TON units. FIG. 10 is a contour plot representing a graphical analysis of the model. In FIG. 10, each concentric line defines a region of equal TON as estimated by the model. The highest TON is estimated from a Nelder-Mead algorithm, Numerical Recipes, William H. Press, Cambridge University Press, Cambridge, England, 1986, pp. 288-293), is 1346.

TABLE 5

| Ti(acac)2 | Cr(acac)3 | Ce(acac)3 |
|---|---|---|
| 20 | 40 | 40 |
| 40 | 20 | 40 |
| 40 | 40 | 20 |

Sample points of the array are selected that contain all TON values within 2.5 standard deviations of the highest TON and correspondingly with a 99% probability of containing the highest TON. In this case, sample points containing all TON above (1346−2.5*33)=263 are selected. These are given in TABLE 6.

TABLE 6

| Ti(acac)2 | Cr(acac)3 | Ce(acac)3 |
|---|---|---|
| 20 | 60 | 60 |
| 60 | 20 | 60 |
| 60 | 60 | 20 |

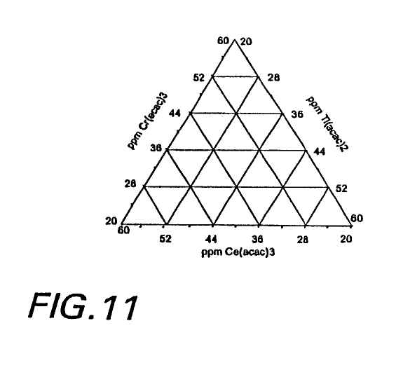

A new simplex is generated for the next experiment in the same fashion as the first, except that the starting and ending points were 20 and 60 ppm rather than 0 and 100 ppm for each component as shown in FIG. 11. The experiment composition and results are given in TABLE 7.

TABLE 7

| Ti(acac)2 | Cr(acac)3 | Ce(acac)3 | TON |
|---|---|---|---|
| 20 | 20 | 60 | 1199 |
| 20 | 28 | 52 | 1395 |
| 20 | 36 | 44 | 1418 |
| 20 | 44 | 36 | 1521 |
| 20 | 52 | 28 | 1387 |
| 20 | 60 | 20 | 1252 |
| 28 | 20 | 52 | 1326 |
| 28 | 28 | 44 | 1514 |
| 28 | 36 | 36 | 1497 |
| 28 | 44 | 28 | 1557 |
| 28 | 52 | 20 | 1328 |
| 36 | 20 | 44 | 1369 |
| 36 | 28 | 36 | 1523 |
| 36 | 36 | 28 | 1549 |
| 36 | 44 | 20 | 1447 |
| 44 | 20 | 36 | 1368 |
| 44 | 28 | 28 | 1541 |
| 44 | 36 | 20 | 1341 |
| 52 | 20 | 28 | 1354 |
| 52 | 28 | 20 | 1374 |
| 60 | 20 | 20 | 1222 |

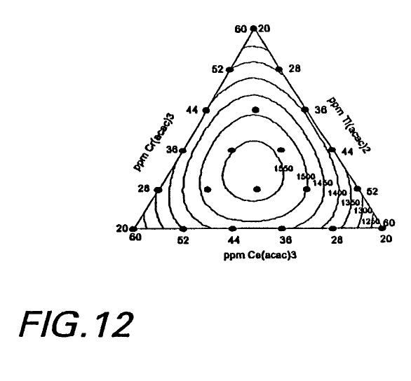

Analysis of the data using regression techniques for mixtures, (Cornell, “Experiments with Mixtures”, John Wiley and Sons, 1990) yields a model that defines TON=+12.68*A+8.768*B+7.60*C−0.270*A*B−0.270*A*C−0.0622* B*C+0.0343A*B*C, where A=ppm Ti(acac)2; B=ppm Cr(acac)3; and C=ppm Ce(acac)3. The contour plot shown in FIG. 12 represents a graphical analysis of the model where concentric lines define regions of equal TON. The highest TON, estimated using the Nelder-Mead algorithm (Numerical Recipes, William H. Press, Cambridge University Press, Cambridge, England, 1986, p 288-293), is 1566 at A=33.1 ppm, B=31.6 ppm, and C=35.3 ppm with a model standard deviation of 40 TON units.

### EXAMPLE 2

This example illustrates an identification of an active and selective catalyst for the production of aromatic carbonates using a procedure according to the invention. The procedure identifies the best cocatalyst from a complex chemical space, where the sampling space is defined as an assemblage of ratios of combinations of certain Group IVb, Group VIb, and Lanthanide Group metal complexes.

The sampling space in this Example consists of the same components as Example 1, TABLE 2. The components are sampled according to a ternary gradient mixture (a simplex) experiment with M=6, n=3, with component proportions ranging from 0 to 100 ppm. The components in this Example are sampled in a compacted space defined by the relationship (100/(n(M−1))) to (100-200/(n(M−1))). The space calculates as (100/(3(6−1)))=6.67 ppm to (100-200/(3(6−1)))=86.67 ppm. The sampling space for each axis divided by the number of intervals minus 1 (M-−1) or ((86.67−6.67)/4) provides interval spacing for each axis.

TABLE 8

| Ti(acac)2 | Cr(acac)3 | Ce(acac)3 | TON |
|---|---|---|---|
| 6.67 | 86.67 | 6.67 | 891 |
| 6.67 | 66.67 | 26.67 | 1082 |
| 6.67 | 46.67 | 46.67 | 1124 |
| 6.67 | 26.67 | 66.67 | 1022 |
| 6.67 | 6.67 | 86.67 | 854 |
| 26.67 | 66.67 | 6.67 | 964 |
| 26.67 | 26.67 | 46.67 | 1389 |
| 26.67 | 6.67 | 66.67 | 948 |
| 26.67 | 46.67 | 26.67 | 1338 |
| 46.67 | 46.67 | 6.67 | 1071 |
| 46.67 | 6.67 | 46.67 | 998 |
| 46.67 | 26.67 | 26.67 | 1267 |
| 66.67 | 26.67 | 6.67 | 999 |
| 66.67 | 6.67 | 26.67 | 950 |
| 86.67 | 6.67 | 6.67 | 893 |

All possible combinations of interval spacing values are postulated as shown in TABLE 8. Virtual postulated combinations (ones that include negative values) have been discarded. The resulting combinations of positive interval spacing values define test samples. Mixture of 1.0 ml. in volume are made up according to the combinations of TABLE 8. A 25 microliter aliquot of each mixture is dispensed into a 2 ml reaction vial, forming a film on the vial bottom. The vials are subjected to reaction at 1000 psi pressure and 100° C. for 120 min. At the end of the reaction time, the system is cooled and depressurized. The contents of each vial are analyzed for diphenyl carbonate product. A turnover number (TON) for each reaction is calculated as mols of diphenylcarbonate/mols of primary catalyst. Results are given in the column TON of TABLE 8.

The data as analyzed using regression techniques for mixtures, (Cornell, “Experiments with Mixtures”, John Wiley and Sons, 1990) yields a model that defines TON =+8.53*A+7.97*B+7.65*C−7.23E−003*A*B−0.0252*A*C+0.0597*B*C +0.015*A*B*C, where A=ppm Ti(acac)2; B=ppm Cr(acac)3; and C=ppm Ce(acac)3. The highest TON is 1391 with a standard deviation of 34 as derived from the Nelder-Mead algorithm (Numerical Recipes, William H. Press, Cambridge University Press, Cambridge, England, 1986, p 288-293).

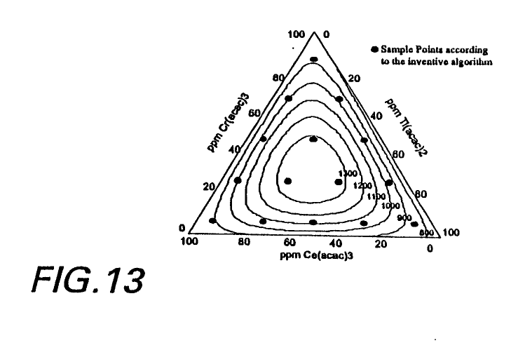

FIG. 13 is a contour plot representing a graphical analysis of the model. In FIG. 13, each concentric line defines a region of equal TON as estimated by the model. The region of highest TON (1300 and above) is in the center of the plot, bounded by the points of TABLE 9.

TABLE 9

| Ti(acac)2 | Cr(acac)3 | Ce(acac)3 |
|---|---|---|
| 26.67 | 26.67 | 46.67 |
| 26.67 | 46.67 | 26.67 |
| 46.67 | 26.67 | 26.67 |

Sample points of the original array which contain all TON values within 2.5 standard deviations of the highest TON are selected. In this case, sample points containing all TON above (1391−2.5*34)=1306 are selected. The compositions are given in TABLE 10.

TABLE 10

| Ti(acac)2 | Cr(acac)3 | Ce(acac)3 |
|---|---|---|
| 20 | 60 | 60 |
| 60 | 20 | 60 |
| 60 | 60 | 20 |

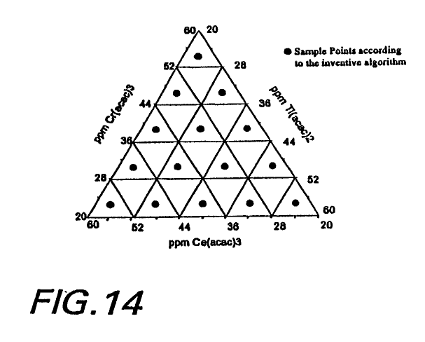

A new simplex is generated for the next experiment in the same fashion as the first, except that the starting and ending points are 20 and 60 ppm rather than 0 and 100 ppm for each component. See FIG. 14. The experiment and results are given in TABLE 11.

TABLE 11

| Ti(acac)2 | Cr(acac)3 | Ce(acac)3 | TON |
|---|---|---|---|
| 22.67 | 22.67 | 54.67 | 1310 |
| 22.67 | 30.67 | 46.67 | 1433 |
| 22.67 | 38.67 | 38.67 | 1471 |
| 22.67 | 46.67 | 30.67 | 1423 |
| 22.67 | 54.67 | 22.67 | 1378 |
| 30.67 | 22.67 | 46.67 | 1419 |
| 30.67 | 30.67 | 38.67 | 1580 |
| 30.67 | 38.67 | 30.67 | 1511 |
| 30.67 | 46.67 | 22.67 | 1452 |
| 38.67 | 22.67 | 38.67 | 1429 |
| 38.67 | 30.67 | 30.67 | 1520 |
| 38.67 | 38.67 | 22.67 | 1486 |
| 46.67 | 22.67 | 30.67 | 1434 |
| 46.67 | 30.67 | 22.67 | 1424 |
| 54.67 | 22.67 | 22.67 | 1268 |

Analysis of the data using regression techniques for mixtures yields a model that defines TON=+7.13*A+12.17*B+10.70*C−0.184*A*B−0.181* A*C−0.293*B*C+0.0348*A*B*C where A=ppm Ti(acac)2; B=ppm Cr(acac)3; and C=ppm Ce(acac)3. The contour plot shown in FIG. 13 represents a graphical analysis of the model where each concentric line defines a region of equal TON. The highest TON is 1556 at A=32.9 ppm, B=34.3 ppm, and C=32.7 ppm with a model standard deviation of 28 TON units.

These Examples show that a lesser number of samples can be used according to the invention, Example 2, to identify the same best results from an experimental space as identified by a method requiring a greater number of samples according to Example 1.

While preferred embodiments of the invention have been described, the present invention is capable of variation and modification and therefore should not be limited to the precise details of the Examples. The invention includes changes and alterations that fall within the purview of the following claims.

What is claimed is:

1. A method of conducting a mixture experiment, comprising:
determining an experimental space comprising n factors and a first factor in M number of factor level intervals and in a range of Aₘᵢₙ to Aₘₐₓ where A is a proportion of the factor level to total factor levels; and
conducting an experiment on the first factor sampled in a range of levels determined according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1))).

2. The method of claim 1, where the experimental space comprises a first factor in 0 to 100 levels and the first factor is sampled in a range of levels determined according to a relationship (100/(n(M−1))) to (100-200/(n(M−1))).

3. The method of claim 1, wherein the factors are components of a catalyst.

4. The method of claim 1, wherein the space comprises the first factor in increments of 1/(M−1) within the range.

5. The method of claim 1, wherein the experimental space comprises a three component mixture and a second factor level range is determined from a minimum level value to a maximum level value according to the relationship (100/(3(M′−1))) to (100-200/(3(M′−1))), where M′ is a number of level intervals for the second component.

6. The method of claim 1, wherein the experimental space comprises a three component mixture and a second factor level range is determined from a new minimum level value to a new maximum level value according to the relationship (100/(3(M′−1))) to (100-200/(3(M′−1))), where M′ is a number of level intervals for the second component and the space comprises the first factor in increments of 1/(M−1) within its range and the second factor in increments of 1/(M′−1) within its range.

7. The method of claim 1, wherein the experimental space comprises a three component mixture and a second factor level range is determined from a new minimum level value to a new maximum level value according to the relationship (100/(3(M′−1))) to (100-200/(3(M′−1))), where M′ is a number of level intervals for the second component and the space comprises the first factor in increments of 1/(M−1) within its range and the second factor in increments of 1/(M′−1) within its range;
and the method further comprises:
determining values for levels of a third factor of the mixture from a positive difference between 1.0 and values for a summation of levels of the other factor levels; and
conducting an experiment on samples defined according to the determined values for each factor.

8. The method of claim 1, wherein the experimental space comprises a four component mixture and a second factor level range is determined from a new minimum level value to a new maximum level value according to a relationship, (100/(3(M′−1))) to (100-200/(3(M′−1))), where M′ is a number of level intervals for the second component.

9. The method of claim 1, wherein the experiment comprises a CHTS experiment.

10. The method of claim 9, wherein the experiment is a CHTS experiment comprising steps of:
preparing a combinatorial library comprising a plurality of reagent compositions according to the experimental space;
effecting parallel reaction of the library to produce products; and
evaluating the products to select a lead from the library of reactants.

11. The method of claim 9, wherein the CHTS experiment comprises providing a reactor plate comprising a substrate with an array of reaction cells containing at least one reactant according to the experimental spaces and reacting the reactant in parallel with other reactants.

12. The method of claim 9, wherein the CHTS comprises effecting parallel chemical reactions of an array of reactants defined according to the experimental space.

13. The method of claim 9, wherein the CHTS comprises effecting parallel chemical reactions on a micro scale on reactants defined according to the experimental spaces.

14. The method of claim 9, wherein the CHTS comprises an iteration of steps of simultaneously reacting a multiplicity of tagged reactants prepared according to the experimental space and identifying a multiplicity of tagged products of the reaction and evaluating the identified products after completion of a single or repeated iteration.

15. The method of claim 9, wherein the experimental space factors comprise reactants, catalysts and conditions and the CHTS comprises
(A) (a) reacting a reactant selected according to the experimental space under a selected set of catalysts or reaction conditions; and (b) evaluating a set of results of the reacting step; and
(B) reiterating step (A) wherein a selected experimental space selected for a step (a) is chosen as a result of an evaluating step (b) of a preceding iteration of step (A).

16. The method of claim 9, wherein the factors include a catalyst system comprising a Group VIIIB metal.

17. The method of claim 9, wherein the factors include a catalyst system comprising palladium.

18. The method of claim 9, wherein the factors include a catalyst system comprising a halide composition.

19. The method of claim 9, wherein the factors include an inorganic co-catalyst.

20. The method of claim 9, wherein the factors include a catalyst system includes a combination of inorganic co-catalysts.

21. The method of claim 9, wherein the factors comprise a reactant or catalyst at least partially embodied in a liquid and effecting the CHTS method comprises contacting the reactant or catalyst with an additional reactant at least partially embodied in a gas, wherein the liquid forms a film having a thickness sufficient to allow a reaction rate that is essentially independent of a mass transfer rate of additional reactant into the liquid to synthesize products that comprise the results.

22. The method of claim 1 wherein the experiment is a CHTS experiment that identifies at least one point comprising an improved result; and the method further
comprises: defining at least one additional experimental space comprising at least one lattice of points representing reaction factor levels in a smaller increment around the point of lead result;
conducting at least a next CHTS experiment on the experimental space to identify at least one point comprising a lead result comprising a set of levels of reaction factors.

23. A method for defining a reduced set of samples for an experimental space and conducting an experiment on the samples,
comprising: determining an experimental space comprising n factors in M number of evenly spaced factor level intervals over a range of Aₘᵢₙ to Aₘₐₓ where A is a proportion of a factor level to total factor levels;
specifying new factor level ranges for each factor according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1)));
selecting samples of combinations of factors in a set of M−1 evenly spaced levels within the specified levels; and
conducting an experiment on the samples.

24. The method of claim 23, comprising selecting only possible combinations of evenly spaced factor levels within the specified ranges.

25. The method of claim 23, comprising selecting all possible combinations of evenly spaced factor levels within the specified ranges.

26. A system for conducting an experiment, comprising;
a reactor for effecting a CHTS method on an experimental space to produce results; and
a programmed controller for the reactor that defines an experimental space comprising a lattice of points representing increments of reaction factor levels from a minimum level value to a maximum level value according to the relationship (Aₘᵢₙ+(Aₘₐₓ−Aₘᵢₙ)/(n(M−1))) to (Aₘₐₓ−(Aₘₐₓ−Aₘᵢₙ)/(n(M−1))) where M is a number of intervals for the factor levels of the range, n is a number of mixture components and A is a proportion of the factor level to total factor levels.

27. The system of claim 26, wherein the controller is a computer, processor or microprocessor.

28. The system of claim 26, further comprising a dispensing assembly to charge factor levels of reactants or catalysts representing the catalyzed chemical experimental space to wells of an array plate for charging to the reactor.

29. The system of claim 26, wherein the dispensing assembly is controlled by the controller to charge factor levels of reactants or catalysts according to the controller defined space.

30. The system of claim 26, further comprising a detector to detect results of the CHTS method effected in the reactor.

31. A system for conducting an experiment, comprising;
a reactor for effecting a CHTS method on an experimental space to produce results; and
a programmed controller for the reactor for inputing experimental space information comprising n factors in M number of factor level intervals and in a range of Aₘᵢₙ to Aₘₐₓ where A is a proportion of a factor level to total factor levels, specifying new factor level ranges for each factor according to a relationship (Aₘᵢₙ+(Aₘₐₓ−Aₘᵢₙ)/(n(M−1))) to (Aₘₐₓ−(Aₘₐₓ−Aₘᵢₙ)/(n(M−1and selecting samples of combinations of factors in a set of M−1 evenly spaced levels within the specified levels for charge to the reactor.
